(19) United States
(12) Patent Application Publication
(10) Pub. No.: US 2012/0219987 A1
(43) Pub. Date: Aug. 30, 2012

(54) DEVICE FOR ELECTROPORATION AND LYSIS

(76) Inventors: Tofy MUSSIVAND, Ottawa (CA); Bashir Iqbal MORSHED, Ottawa (CA); Maitham SHAMS, Ottawa (CA)

(21) Appl. No.: 13/324,327

(22) Filed: Dec. 13, 2011

Related U.S. Application Data
(60) Provisional application No. 61/422,532, filed on Dec. 13, 2010.

(51) Int. Cl. C12M 1/42; G01N 33/52; C12N 13/00

(52) U.S. Cl. 435/40.5; 435/306.1; 435/173.7

(57) ABSTRACT

There is provided a membrane disruption device including a sample container and an electrode assembly. The sample container includes a sample-containing space defined by a containing surface and configured for containing a cellular-material comprising fluid, wherein the sample-containing space includes at least one membrane disruption space. The electrode assembly including a first electrode portion spaced apart from a second electrode portion, for generating an electric field within the membrane disruption space and effecting membrane disruption of a cell of a cellular material-comprising fluid disposed within the membrane disruption space.

### CROSS-REFERENCE TO RELATED APPLICATIONS

This application claims the benefit of priority under 35 U.S.C. 119 (e) to U.S. Provisional Patent Application Ser. No. 61/422,532 filed Dec. 13, 2010, the subject matter of which is incorporated by reference in its entirety.

### FIELD

The present disclosure relates to methods and devices for disrupting the membranes of cells and, in particular, the electroporation and lysis of cells.

### BACKGROUND

Cell lysis is defined as the breaking open of a cell by decomposition of the cell membrane using an external stimulus [1]. Cell lysis has become a very useful technique in biochemistry and medicine. Cell lysis allows for the isolation of proteins, genetic material, and cellular organelles, all of which are used in studying the functions of different types of cells. Techniques for achieving cell lysis include optical, mechanical, acoustic, chemical, and electrical methods [2]. Lysis using a high electric field is defined as electrical lysis, or dielectric breakdown [3].

Electroporation is the phenomenon of creating pores (poration) through the cell membrane under the influence of an applied electric field [4]. Cell poration allows for the introduction of genetic material or antibiotics into the cell through re-sealable pores that are introduced into the cell membrane. The introduction of genetic material into cells has been proven to be useful in genetic studies, such as gene silencing, expression, and cloning [5]. Cell poration has also been identified as a means for administering antibiotics directly into target cells.

When the electric field is applied to a cell, a large amount of charges accumulate along the membrane creating a large potential across the membrane [6]. This transmembrane potential is much larger than the action potential of a cell and causes an electrical force on the phospholipids of the membrane. This electrical force causes the phospholipids to reorient. If the electrical field is increased such that the transmembrane potential reaches a critical value, the phospholipids of the membrane undergo a conformational change which causes the formation of pores. The critical transmembrane potential for the formation of these pores is approximately 1 V [7,8]. The electric field required to initiate the formation of the pores is approximately 0.1 to 0.15 MV/m and is called the threshold electric field [3]. After the initiation of pore formation (nucleation period), the pores expand with time [7]. If the electric field is sustained, the average sizes of the pores increase. If the electric field is removed before the pores reach a critical diameter, the pores will reseal. Electrical lysis occurs when the electric field is applied for a period allowing the pore diameters to reach the critical pore diameter, approximately 40 nm [9,10]. Once the pores reach the critical diameter they begin to expand spontaneously which results in the destruction of the cell membrane, causing electrical lysis. However, if the electric field is withdrawn prior to critical pore diameter, pores reseal and demonstrate the electroporation phenomenon.

### SUMMARY

In one aspect, there is provided a membrane disruption device including a sample container and an electrode assembly. The sample container includes a sample-containing space defined by a containing surface and configured for containing a cellular-material comprising fluid, wherein the sample-containing space includes at least one membrane disruption space. The electrode assembly includes a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space. When the device is disposed in an orientation for containing a cellular material-comprising fluid, each one of the first electrode portion and the second electrode portion, independently, either: (i) defines a lowermost portion of a containing surface portion of the sample container, and the lowermost portion is disposed in a vertical plane orthogonally intersecting the longitudinal axis of the sample-containing space, or (ii) is disposed, relative to a lowermost portion of a containing surface portion of the sample container, vertically above the lowermost portion by a distance of less than one (1) wherein the lowermost portion is disposed in a vertical plane orthogonally intersecting the longitudinal axis of the sample-containing space.

In another aspect, there is provided a membrane disruption device including a sample container and an electrode assembly. The sample container includes an internal surface defining a sample-containing space configured for containing a cellular-material comprising fluid, wherein the sample-containing space includes at least one membrane disruption space. The electrode assembly includes a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space. The internal surface includes the first and the second electrode portions.

In another aspect, there is provided a membrane disruption device including a sample container and an electrode assembly. The sample container includes an internal surface defining a sample-containing space configured for containing a cellular-material comprising fluid, wherein the sample-containing space includes at least one membrane disruption space. The electrode assembly includes a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space. A fraction of the internal surface defines the first and the second electrode portions.

In another aspect, there is provided a membrane disruption device including a device substrate and an electrode assembly. The device substrate includes a cavity, wherein a sample-containing space is defined within the cavity, and wherein the sample-containing space is configured for containing a cellular-material comprising fluid, wherein the sample-containing space includes at least one membrane disruption space. The electrode assembly includes a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space. The first and the second electrode portions are embedded in the device substrate.

In another aspect, there is provided a membrane disruption device including a sample container, an electrode assembly, and at least one supply port. The sample container includes a sample-containing space defined by a containing surface and configured for containing a cellular-material comprising fluid, wherein the sample-containing space includes at least one membrane disruption space. The electrode assembly includes a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space. Each one of the at least one supply port is disposed in fluid communication with the membrane disruption space for effecting supply of material into the membrane disruption space from vertically above the sample-containing compartment.

In another aspect, there is provided a method for staining a cellular sample for visualisation in microfluidic applications. The method includes: a) combining a cell sample with a stain, and b) effecting centrifugal separation of the stained cell-sample and removing at least a fraction of the resulting supernatant.

### DESCRIPTION OF THE DRAWINGS

These and other features of the preferred embodiments will become more apparent in the following detailed description in which reference is made to the appended drawings wherein:

FIG. 1 is a schematic view from the top of an embodiment of a device.

FIG. 2 is an exploded view of an embodiment of a device. All four layers (top glass slide, microchannel, integrated electrodes, and bottom glass slide) of the device are shown separated and vertically aligned;

FIG. 3 is an expanded view of a portion of FIG. 1 labelled A;

FIG. 4 is a detailed fragmentary view of the electrode structure illustrated in FIG. 3;

FIG. 5 is a cross-sectional view of the device shown in FIG. 3 through the line 1-1;

FIG. 6 is a cross-sectional view of the device shown in FIG. 3 through the line 2-2;

FIG. 7 is identical to FIG. 5, and illustrates the disposition of the electrode assembly relative to the sample container; and

FIG. 8 is a schematic diagram depicting the electrical connection of the device with the pulse generator or the signal administrating module;

### DETAILED DESCRIPTION

In the present application, where an element is introduced with “a”, “an” or “the” it should be understood as extending to a plurality unless the context dictates otherwise.

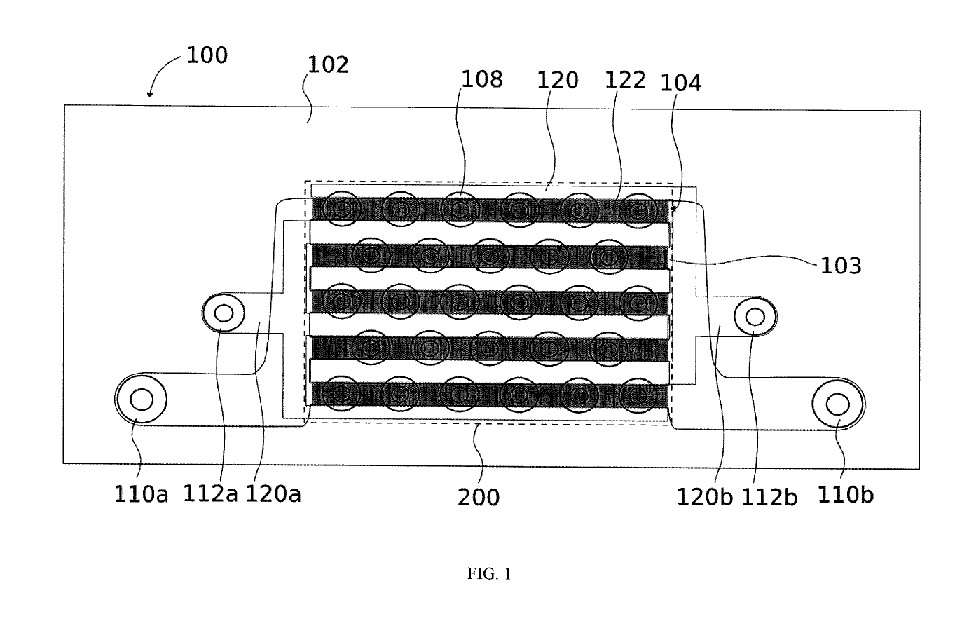

Referring to FIGS. 1 to 8, there is provided a membrane disruption device 100 for applying an electric field to a cell sample. While the device 100 may be referred to as membrane disruption device, the device can be used both to electroporate cells or, upon application of an appropriate electric field, to lyse cells. Collectively, these effects can be referred to as membrane disruption. While the device can be used with biological cells, as will be apparent to a person of skill in the art, it may also be applicable to other objects having an inner conducting core surrounded by a dielectric layer, e.g. a lipid bilayer membrane.

In some embodiments, the device 100 is a portable device. In some embodiments, the device 100 is a handheld device. In some embodiments, the device 100 is a reusable device.

The membrane disruption device includes a container 103. The container 103 includes a sample-containing space 1042 defined by a containing surface 1044 and configured for containing a cellular-material comprising fluid. The sample-containing space 1042 includes at least one membrane disruption space 200. The shape and configuration of the membrane disruption space 200 is not specifically restricted and various shapes and configurations are within the scope of the present invention. The space 200 may be defined by a sealed compartment accessible by single or multiple ports, or it may be an open-topped or open ended space.

In some embodiments, for example, the sample-containing compartment includes a microchannel 104, such that the device includes one or more microchannels. In the illustrated embodiment, there are five microchannels 104a to 104e. In some embodiments, for example, the microchannel 104 includes a minimum width, measured horizontally in a plane orthogonal to the longitudinal axis 1046 of the microchannel 104, of between 10 μm and 10 mm. In some embodiments, for example, the microchannel 104 includes a minimum width “W”, measured horizontally in a plane orthogonal to the longitudinal axis of the microchannel, of between 10 μm and 600 μm. In some of these embodiments, for example, the minimum width “W” is between 40 μm and 600 μm. For example, in some embodiments, the width is 400 μm. In some embodiments, for example, the microchannel 104 includes a minimum length of 100 μm. In some embodiments, the minimum length of the microchannel 104 is between 100 μm and 20 millimetres. For example, in some embodiments, the minimum length of the microchannel 104 is between 100 μm and 12 millimetres.

In some embodiments, for example, the microchannel 104 is defined on one or more substrates. In some embodiments, for example, each one of the one or more substrates includes a substantially optically transparent portion. In some embodiments, for example, each one of the one or more substrates is a glass slide. In some embodiments, for example, the substrate includes a pair of glass slides 102a, 102b, wherein the microchannel 104 is etched into one or both of the glass slides, and the pair of glass slides is then subsequently fused. In an example embodiment, the dimensions of each one of the glass slides 102a, 102b is 15 mm long, 3 mm wide and 1.1 mm thick and the dimensions of a microchannel 104 are 400 μm in width, 40 μm in depth, and 12 mm in length.

The device 100 also includes an electrode assembly 300. The electrode assembly 300 includes a first electrode 114 and a second electrode 116, wherein the first electrode 114 is spaced apart from the second electrode 116. The first electrode 114 includes one or more first electrode portions 114a. The second electrode 116 includes one or more second electrode portions 116a. When the electrode assembly 300 is coupled to a power source (such as a current or voltage source) which effects an electric potential difference between the first electrode portion 114a and the second electrode portion 116a, an electric field is generated within the membrane disruption space 200 and effects membrane disruption (such as disruption of a lipid bilayer) of a cell of a cellular material-comprising fluid disposed within the membrane disruption space 200.

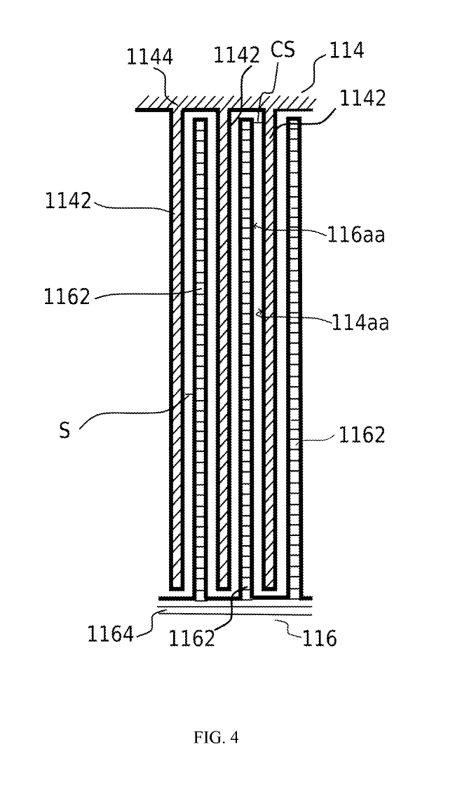

In some embodiments, for example, when the electrode assembly 300 is coupled to a power source such that an electric potential difference is effected between the first electrode portion 114a and the second electrode portion 116a, the electric field is generated between the first electrode portion 114a and the second electrode portion 116a and the membrane disruption of a cell is effected when the cell is disposed between the first electrode portion 114a and the second electrode portion 116a. In some embodiments, each one of the first electrode portion 114a and the second electrode portion 116a is disposed within the membrane disruption space 200. In some embodiments, and referring to FIG. 4, the first electrode portion 114a is spaced apart from the second electrode portion 116a by a spacing distance “S” of less than 200 μm. In some embodiments, for example, the spacing distance “S” is less than 100 μm. In some embodiments, for example, the spacing distance “S” is 10 μm or less.

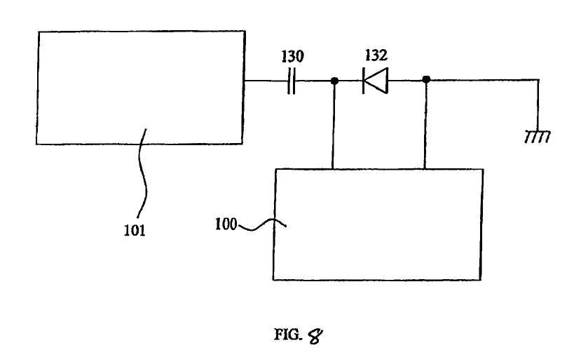

In some embodiments, for example, and referring to FIG. 7, each one of the first electrode portion 114a and the second electrode portion 116a is co-operatively disposed relative to the container 103 such that, when the sample-containing space 1042 is containing its maximum capacity of a cellular material-comprising fluid 500, each one of the first electrode portion 114a and the second electrode portion 116a is disposed vertically below an upper surface portion 400 of the contained cellular material-comprising fluid 500 by a distance L1 of at least 30 μm. In some embodiments, for example, the distance L1 is at least 40 μm. In some embodiments, for example, the distance L1 is between 30 μm and 100 μm. In some embodiments, for example, the distance L1 is between 40 μm and 100 μm.

In one aspect relating to the disposition of the electrode assembly 300 relative to the container 103, when the device 100 is disposed in an orientation for containing a cellular material-comprising fluid, each one of the first electrode portion 114a and the second electrode portion 116a, independently, either: (i) defines a lowermost portion 1044a of a containing surface portion of the sample container 103, and the lowermost portion 1044a is disposed in a vertical plane orthogonally intersecting the longitudinal axis of the sample-containing space, or (ii) is disposed, relative to a lowermost portion of a containing surface portion of the sample container 103, vertically above the lowermost portion by a distance of less than one (1) μm, wherein the lowermost portion is disposed in a vertical plane orthogonally intersecting the longitudinal axis of the sample-containing space.

The sample container 103 includes an internal surface 1045 that defines the sample-containing space 1042. In this respect, in another aspect, the internal surface 1045 includes the first and the second electrode portions 114a, 116a. In this respect, in a further aspect, a fraction of the internal surface 1045 defines the first and the second electrode portions 114a, 116a.

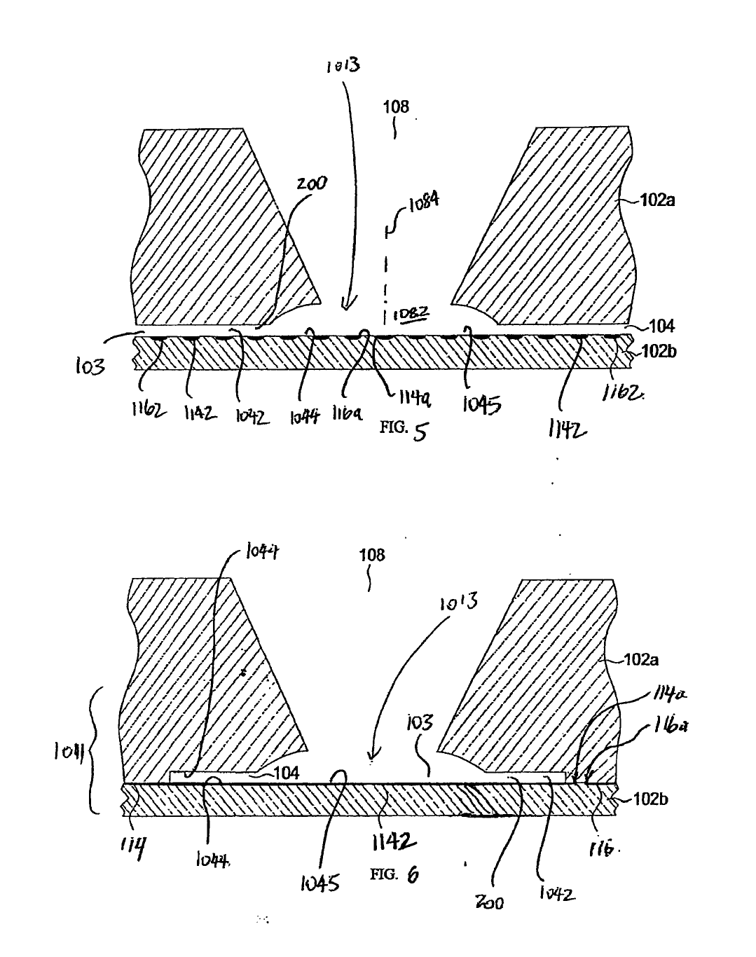

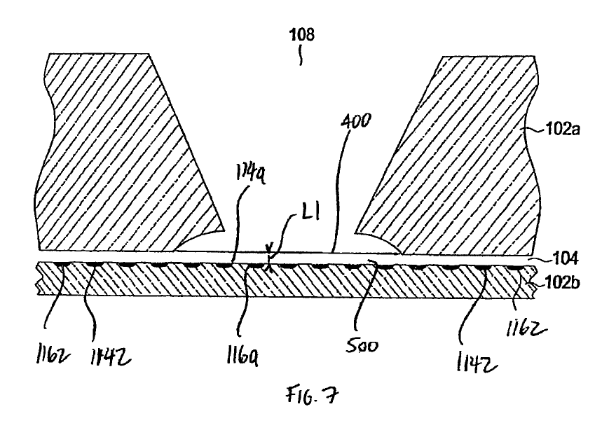

In another aspect, and referring to FIGS. 5 and 6, the membrane disruption device 100 includes a substrate or an assembly of substrates (hereinafter, either of these is referred to as the “device substrate 1011”). The device substrate 1011 includes a cavity 1013, wherein the sample-containing space 1042 is defined within the cavity 1013, and the first and the second electrode portions 114a, 116a are embedded in the device substrate 1011.

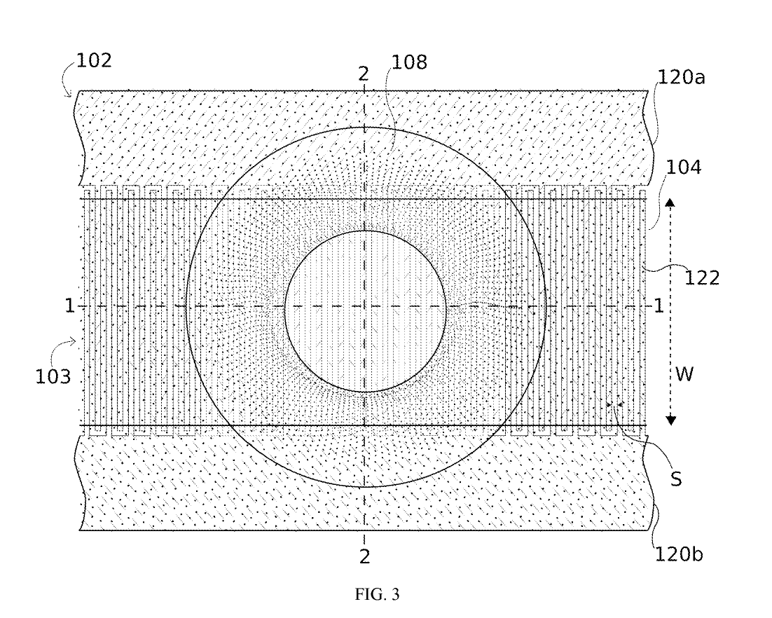

In some embodiments for example, the first electrode 114 includes an operative first electrode section 114aa, and the second electrode 116 includes an operative second electrode section 116aa. Each one of the operative first electrode section 114aa and the operative second electrode section 116aa includes a surface area of at least 500 μm². In some embodiments, each one of the operative first electrode section 114aa and the second electrode section 116aa includes a surface area of at least 2000 μm². Each portion of the operative first electrode section 114aa is a first electrode portion 114a. In this respect, the operative first electrode section 114a is defined by a continuous arrangement of a plurality of first electrode portions 114a. Each portion of the operative first electrode section 116aa is a second electrode portion 116a. In this respect, the operative second electrode section 114a is defined by a continuous arrangement of a plurality of second electrode portions 114a. In some embodiments, for example, each portion of the operative first electrode section is spaced apart from the second electrode by a closest spacing distance “CS”. The closest spacing distance “CS” is less than 200 μm. In some embodiments, for example, the closest spacing distance “CS” is less than 100 μm. In some embodiments, for example, the closest spacing distance “CS” is 10 μm or less. In some embodiments, and referring to FIG. 3, each one of the operative first electrode section 114aa and the operative second electrode section 116aa extends from a sidewall of the microchannel and across at least 60% of the width W of the microchannel.

With respect to the operative first and second electrode sections 114aa, 116a, in one aspect relating to the disposition of the first and second electrode sections 114aa, 116aa relative to the container 103, when the device 100 is disposed in an orientation for containing a cellular material-comprising fluid, each one of the operative first electrode section 114aa and the operative second electrode section 116aa, independently, either: (i) defines a lowermost portion 1044a of a containing surface portion of the sample container 103, and the lowermost portion 1044a is disposed in a vertical plane orthogonally intersecting the longitudinal axis of the sample-containing space, or (ii) is disposed, relative to a lowermost portion of a containing surface portion of the sample container 103, vertically above the lowermost portion by a distance of less than one (1) μm, wherein the lowermost portion is disposed in a vertical plane orthogonally intersecting the longitudinal axis of the sample-containing space.

Also with respect to the operative first and second electrode sections 114aa, 116a, the sample container 103 includes an internal surface 1045 that defines the sample-containing space 1042. In this respect, in another aspect, the internal surface 1045 includes the operative first and second electrode sections 114aa, 116aa. In this respect, in a further aspect, a fraction of the internal surface 1045 defines the operative first and second electrode sections 114aa, 116aa.

Also with respect to the operative first and second electrode sections 114aa, 116a, in another aspect, and referring to FIGS. 5 and 6, the membrane disruption device 100 includes a substrate or an assembly of substrates (hereinafter, either of these is referred to as the “device substrate 1011”). The device substrate 1011 includes a cavity 1013, wherein the sample-containing space 1042 is defined within the cavity 1013, and the operative first and second electrode sections 114aa, 116aa are embedded in the device substrate 1011.

In some embodiments, for example, the electrode assembly 300 includes an interdigitated electrode structure 122, and the interdigitated electrode structure 122 includes both of the first and second operative electrode sections 114aa, 116aa. In some embodiments, for example, the sample-containing compartment is a microchannel 104, and the first and second electrode sections 114aa, 116aa are disposed on opposite sides of the microchannel 104. In some embodiments, for example, each one of the operative first electrode section 114aa and the operative second electrode section 116aa extends across substantially the entire width of the microchannel 104.

In some embodiments of the interdigitated electrode structure 122, each one of the electrode sections 114aa, 116aa includes a respective digitated portion 1142,1162 that includes a plurality of digits extending from a respective thick (or “base”) electrode portion 1144,1164. In some embodiments, the operative first electrode section 114aa and the operative second electrode section 116aa of the interdigitated electrode structure are coplanar or substantially coplanar. In some embodiments, the thickness of the thick electrode portion 1144 (1164) is at least 100 μm. In some embodiments, for example, the thickness of the thick electrode portion 1144 (1164) is 500 μm. In some embodiments, the thickness of the digits of the digitated portion 1142 (1162) is at least one (1) μm. In some embodiments, for example, the thickness of the digits of the digitated portion 1142 (1162) is at least 10 μm.

In some embodiments of the interdigitated electrode structure 122, the operative first and second electrode sections 114aa, 116aa of the interdigitated electrode structure are positioned parallel to the microchannels 104 and on a respective one of the two sides of the microchannel 104 to provide alternative polarity of charge (negative or positive) when electrically connected to the power source 101. In some embodiments, for example, this electrode configuration provides for minimal voltage drop from the electrode ports 112a, 112b (see below) to the digits 1142, 1162 of the operative first and second electrode sections 114a, 114b. In some embodiments, for example, the width of the digits 1142, 1162 of the operative first and second electrode sections 114aa, 116aa, is equal to the gap between these digits. In some embodiments of the above-described arrangements of electrodes, generation of maximum electric fields is promoted within the parallel microchannels, and such high electric field spreads up to 50% of the surface area of the bottom surface in the membrane disruption space. The developed electric field is more evenly distributed compared to other possible arrangements, maximizing the probability of randomly oriented cells within the membrane disruption space 200 being electroporated or lysed. In some embodiments, the operative first and second electrode sections 114aa, 116aa are suitably formed on the bottom surface of the microchannel 104 and thereby effect generation of a higher electric field at the bottom of the microchannel 104, where cells settle. In some embodiments, the electrode sections 114aa, 116aa occupy the entire length of a microchannel 104. In other embodiments, they occupy a portion of the length. In some embodiments, each digit, of the electrode sections 114aa, 116aa, extends the entire width of a microchannels. In other embodiments, it extends over a portion of the width.

In some embodiments, for example, the minimum width of a microchannel 104 can be related to the cell sizes in the samples, as the minimum width needs to be larger than the maximum cell size. For example, for human buccal samples, the minimum width can be 40 μm. Smaller dimensions, in this case, increase the tendency of cell adherence and reduce ease of washing of the micro-device for reuse. The maximum width of a microchannel 104 is limited by fabrication constraints, as well as increased resistivity of the interdigitated electrode structure 122. In some embodiments, the depth of the microchannel 104 is also limited by fabrication constraints. For some applications, microchannel depths of less than 40 μm are less effective because a depth of greater than 40 μm is needed to reduce cell adherence on the surface. While not excluded from the scope of the present invention, microchannel depths of more than 100 μm are less effective where the electrode portions 114a, 116a are disposed at or near the bottom of the microchannel, as the higher electric field is generally confined to the bottom portion of the microchannel and cells residing about the top portion of the microchannel 104 might not be subjected to sufficient electric field to effect membrane disruption. In one embodiment, the microchannel depth is 40 μm. In some embodiments, for a cell diameter D, the cross-cross sectional area of the membrane. In some embodiments, the minimum cross-sectional area of the microchannel 104 is 1600 μm². In some embodiments, the maximum cross-sectional area of the microchannel 104 is 100,000 μm². The length of a microchannel 104 is design specific, with suitably a minimum of 40 μm and a maximum constrained by the device length. In an example embodiment, the length of the parallel microchannel is 12 mm.

In some embodiments, the configuration of the electrode sections 114aa, 116aa positioned in the parallel microchannels 104a-104e maximizes the area of the effective electric field and minimize excitation voltage. In some embodiments, each digit of the electrode sections 114aa, 116aa of the interdigitated electrode structure includes a width of 10 μm or less, or the minimum allowed by available technology, which ensures the maximization of area of the effective electric field. For example, the digits of the electrode sections 114aa, 116aa are spaced apart from one another by a closest spacing distance of 10 μm or less, or the minimum allowed by available technology, which ensures the lowest possible excitation voltage to develop a sufficient electric field to effect membrane disruption. The dimensions, and spacing distance between the electrodes are provided here for exemplary purposes. Smaller dimensions achievable with newer technologies are also encompassed within the scope of this patent. The lower voltage requirements of the device 100 eliminates hazards and expertise required to handle high voltage power supplies typical of the prior art.

In some embodiments, the device 100 includes one or more sample-loading (or “supply”) ports 108 provided for effecting supply (or “loading”) of cell samples to the membrane disruption space. In some of these embodiments, the sample-loading ports are, optionally, used for collection of cell samples. In some embodiments, while the device 100 is suitable for use with invasively or non-invasively collected cell samples, the device 100 can be suitable for use with non-invasively collected cell samples. Methods for sample collection include taking cheek swabs, or by rubbing a finger (or other skin samples) over the sample-loading ports 108 and collecting the shredded cells directly into the microchannels 104. The surface of the device 100 containing the array of sample-loading ports 108 creates an uneven or rough surface (due to the exposed sample loading ports 108 on the surface of the glass slide) and shredded cell debris, when rubbing a finger or other skin samples, accumulate inside the sample-loading ports. In some embodiment, the sample-loading ports 108 are disposed relative to the membrane disruption space 200 in a compact format at a minimum spacing that is allowed by the technology, thus maximizing collection of shredded cells. Furthermore, the area covered by sample-loading ports is suitably designed to match the surface area of a finger or index finger of an average size adult person to enable ease of rubbing.

The supplied cells are admixed with any suitable fluidic carrier, referred to herein as a buffer. The buffer is any fluidic medium that will maintain the proper physiological conditions required for the cells to suspend and will not cause stress to the sample cells. Suitable buffers include, for example, distilled water and Dulbecco's Phosphate-Buffered Saline (DPBS), although other suitable buffers will be known to persons of ordinary skill in the art. In some embodiments, the device includes separate fluidic ports 110 (another typoe of “supply” port) for supplying buffers, cleaning or other fluids for other purposes such as initial wetting of microchannel surfaces. While device 100 suitably includes separate sample-loading port(s) 108 and fluidic port(s) 110, as will be apparent to a person of skill in the art, one port could operate for loading both sample and buffers. In some embodiments, the device 100 includes multiple fluidic ports. In some embodiments, a fluidic port is provided for and fluidly coupled to each one of the plurality of microchannels 104a to 104e. In some embodiments, and as shown in FIG. 1, there are two fluidic ports 110a, 110b located on both sides of each one of the microchannels 104a to 104e. The connection of the microchannels 104a to 104e to the fluidic ports 110 allows for the introduction of buffer into all the microchannels 104a to 104e simultaneously. The type of buffers used is dependent on the type of cells being lysed or electroporated.

In one aspect related to the disposition of the supply ports (ie. sample loading 108 or other fluidic port 110), each one of the at least one supply port is disposed in fluid communication with the membrane disruption space for effecting supply of material into the membrane disruption space from vertically above the sample-containing compartment. This facilitates for easier collection and supply of cell sample to the membrane disruption space and also minimizes time required to provide a cell sample to the membrane disruption space. In some embodiments, for example, the supply port is fluidly coupled to the membrane disruption space with a fluid passage 1082 including a fluid passage axis 1084, wherein the distance along the fluid passage axis is less than 1.1 millimeters.

In some embodiments, the device 100 further includes an electrical connection (not shown) through the electrode ports 112a, 112b for connecting the electrodes 114, 116 to the power source 101 realized with a control circuitry, a pulse power supply or a signal generator that is operated using a battery or a wall power supply. In some embodiments, electrode ports 112 are used to connect the electrodes 114, 116 to the power source 101, that provides electrical pulse of various magnitudes, durations, and number of repetitions, as needed to inflict various degrees of electroporation and lysis on sample cells. This is done by inserting external electrodes (not shown) into the electrode ports 112 and fixing the electrodes within the ports 112. The fixing of electrodes can be done using a silver epoxy or other conductive adhesive. The power source 101 may be part of the device or the device may be adapted for connection to an external power source 101. In some embodiments, the power source includes a battery. The device 100 can be operated with any power source meeting the voltage, current and power requirements.

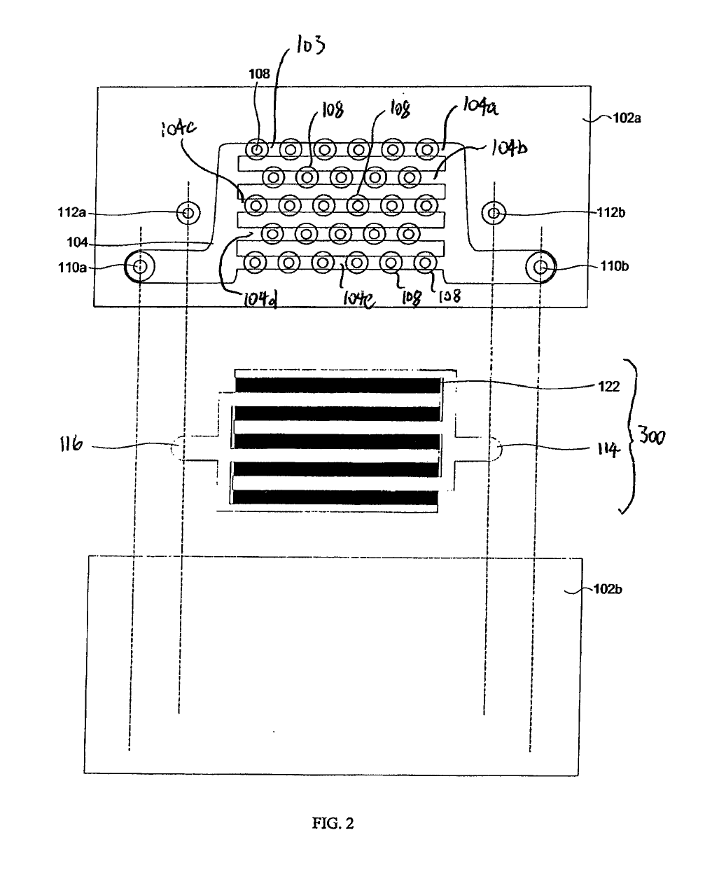

In some embodiments, the device 100 is a reusable, portable, optionally handheld, micro-device for the single cell electrical lysis or electroporation of collected cells. Suitably, the micro-device is a bioelectric chip fabricated using biological microelectromechanical system (BioMEMS) technology and uses microfluidic technology. As described above, the device is suitably fabricated on a substrate 102 including two glass slides (FIG. 2). Through holes are formed in top glass side 102a to provide access ports such as described above, i.e. sample loading ports 108, fluidic ports 110 and electrode ports 112. The bottom surface of the top glass slide is suitably chemically etched to form microchannels 104a to 104e. The top surface of the bottom glass slide 102b contains integrated electrode sections 114aa, 116aa deposited by a suitable fabrication process. In some embodiments, for example, the electrode sections 114aa, 116aa are deposited in etched cavities on the bottom of the microchannel 104. An exemplary manner of depositing the electrode sections 114aa, 116aa in etched cavities on the bottom of the microchannel is as follows. As a first step, the bottom surface of the microchannel 104 (i.e. the top surface of the bottom glass-slide) is suitably etched with chemicals to create cavities that are patterned using a mask for the integrated electrodes with a depth of “h”. In some embodiments, for example, “h” is 100 nm. Then, electrode materials are deposited in these cavities using the exact same mask. The first layer of each of the electrode sections 114aa, 116aa is a binding metal layer, such as Titanium (Ti), of less than half of the cavity depth “h”, for example 20 nm thick. The top layer is metallic material, such as Platinum (Pt), that is inert relative to the sample that is anticipated to be provided in the microchannel 104. The top layer extends for the rest of the cavity depth, for example 80 nm thick. In some embodiments, for example, the total thickness of the electrode sections 114aa, 116aa match the depth of the etched cavity, resulting in the bottom layer of the microchannel 104 to be at the same level of the top layer of the electrode sections 114aa, 116aa, i.e. there would be no vertical difference between the top surface of the electrode sections 114aa, 116aa and the bottom surface of the formed microchannels (see FIG. 7). The glass slides are suitably fused together at a very high temperature to form the device 100. After device fabrication, fluidic port assembly can be coupled to the fluidic ports so as to connect the fluidic ports with micropumps (not shown). The electrode ports 112a, 112b are connected to the power source 101 that administers pulse generation (FIG. 8). To improve protection against accidental damage and reliable operation, a capacitor 130 and a diode 132 may be additionally connected, although these additions are optional as can be understood by a person of skill in the art.

While specific preparation of the cells is not required for use in the device of the present invention, in certain circumstances, it may be desirable to stain the cell samples to aid visualization. In this regard, the present inventors have developed a method of staining cell samples that has particular advantages in the context of micro-device applications. In particular, cell samples prepared by the methods of the present invention can produce a superior yield and/or less adherence than cells prepared by known methods, as described further below.

In another aspect, there is provided a method of effecting membrane disruption of a cell using the device 100. The method involves loading a buffer and cell sample into the microchannel 104 and applying an electric field to the cell sample. In some embodiments, the cell sample is loaded directly into the microchannel 104, e.g. by a sample-loading port 108 in above the microchannel 104. The buffer is loaded at a fluidic port 110 and flows to the microchannel 104 prior to cell sample loading.

The device 100 can be used for membrane disruption in the form of electrical lysis or electroporation without lysis of cell samples by varying the excitation parameters. Both electrical lysis and electroporation are achieved through the development of a high electric field, and both processes may occur in the device 100. The administration, strength and duration of the pulse are controlled by control circuitry, control chip, or an electronic controller. As part of the control circuitry, a capacitor 130 and a reversed biased diode 132 are suitably included, as depicted in the schematic diagram in FIG. 8. The capacitor 130 prevents any accidental damage to the device by blocking any offset DC voltage, while allowing the electric pulse to be transmitted. The reversed biased diode 132 is included to limit the reverse voltage developed across device 100 after the application of the pulse.

In a method for eletroporation of the cell without lysis, suitably a voltage of between about 5 V and about 20 V is applied. In some embodiments, this electroporation is performed with a pulse duration from 1 μs to 100 ms. In some embodiments, the number of pulses is one or many, up to thousands. While the method may be used for various purposes, as will be apparent to a person of skill in the art, in one application, the method of electroporation can be used for the introduction of cellular material into the cell, including genetic material or antibiotics.

In some embodiments, electroporation is performed so as to obtain electrical lysis of cells. For example, to obtain cell lysis, a voltage of between about 15 V and 25 V, preferably 20V, is applied to the cell sample. In some embodiments, this electrical lysis is performed with a pulse duration of between about 3 seconds and about 7 seconds. In some embodiments, the pulse duration is about 5 seconds. Suitably, this pulse has a power of approximately 0.4 W and energy of 2 J. The power consumption and the energy spent are related to pulse, device and buffer parameters, in particular, the pulse amplitude, duration, number of repeats, and the resistivities of the electrodes and buffer fluids. In one embodiment, a single pulse is applied. While the method of cell lysis may be used for various purposes, as will be apparent to a person of skill in the art, in one application, the method of cell lysis can be used for the isolation of cellular components, for example, genetic material.

Known protocols for cell staining particularly with Haematoxylin and Eosin require that the cells are attached on a rigid surface, generally glass. This is due to the fact that one of the steps of the staining protocol is to wash the excess stains with free-flow of deionised water. If the cells are not attached to the surface, the yield reduces significantly as cells are washed away from the working area with the washing fluid. Such cellular attachments create a problem within the micro-device area because cells are required to move freely through the microchannels inside the micro-device and cells having adherent chemicals on their surfaces might result in increased adherence of the cells to the microchannel walls.

While specific preparation of the cells is not required for use in the device 100, in certain circumstances, it may be desirable to stain the cell samples to aid visualization. In this regard, a method of staining cell samples is provided that has particular advantages in the context of micro-device applications. In particular, cell samples prepared by these methods can produce a superior yield and/or less adherence than cells prepared by known methods, as described further below.

In this respect, another aspect provided is a method for staining a cellular sample for visualisation in microfluidic applications. A method for staining a cellular sample for visualisation in microfluidic applications is provided comprising: a) combining a cell sample with a stain; b) effecting centrifugal separation of the stained cell-sample; and c) removing at least a fraction of the resulting supernatant.

In some embodiments, this method includes the steps of i) optionally centrifuging a cell sample suspended in deionised water and discarding the resulting supernatant to increase the concentration of cells to be stained; ii) combining the sample with a stain in solution; iii) adding deionised water after a certain period (rest time) dependant on cell sample type and stain; and iv) centrifuging the stained cell sample and discarding the resulting supernatant. Steps iii) and iv) may be repeated one or more times, as described further below. The method can further include the steps of: v) combining the stained cell sample with a second stain, for example, a counterstain; vi) adding deionised water; and vii) centrifuging the counterstained cell sample and discarding the resulting supernatant. Steps vi) and vii) may be repeated one or more times.

Cell staining can be useful for the identification of parts of lysed cells. A methodology suitable for staining a cell sample collected either invasively or non-invasively involves collecting the cells, mixing with deionised-water and then staining, with centrifugation and removal of supernatant. In some embodiments, the sample is repeatedly mixed with deionised water, centrifuged and the supernatant discarded. The number of times the sample is suspended in deionised water and centrifuged is not particularly restricted, with increased repetition removing more excess cell stain. In some embodiments, these steps are repeated three or more times. In some embodiments, this protocol is followed first with a stain and then with a counterstain. In one embodiment, the stain is Haematoxylin and the counterstain is Eosin. This staining protocol provides a method to stain samples not attached to a surface, while prior art methods are directed to attached cells. While an exemplary protocol is provided below in Example No. 1, it should be noted that the volumes mentioned can be proportionally increased or decreased depending on the sample cell volume. Further, the rest time(s) can be omitted or varied as determined by a person of skill in the art. Further, the rotation speed (rpm) and duration can be varied depending on the cell type and such optimization is within the purview of a person of skill in the art. Cells prepared by the method of the present invention may be used immediately or stored for future use.

The samples resulting from the above-described staining protocol are particularly suitable for micro-device (biochip) applications, as the cells have a lower tendency to adhere to walls of a microchannel of a micro-device. As will be apparent to a person of skill in the art, this is a particular advantage, as it results in easier cleaning of the micro-device for reuse. While, this staining method has particular advantages in the context of biochips, the applications are not particularly restricted and other applications may be apparent to those of skill in the art.

### Example No. 1

Cells were stained for the purpose of testing and the performance of a biochip embodiment of the invention. Cells were prepared for use in electroporation using the following methodology: Freely flowing cells were collected in a 2 mL size centrifuge tube with 1.5 ml deionised-water (D-H₂O). The tubes were centrifuged in a centrifuge machine at low speed (2000 rpm for 5 minutes was found to be suitable.) 1 mL of supernatant was discarded from the top of the tube, leaving about 0.5 mL fluid in each tube. 20 μL of Haematoxylin was added and the tube was gently shaken to diffuse the stain evenly in the fluid. The tube was then allowed to rest for approximately 7 minutes, at which time 1 mL of D-H₂O was added. The tube was then centrifuged again at low speed (2000 rpm for 5 minutes and 1 mL of supernatant was then discarded. The addition of D-H₂O, centrifugation and discarding of the supernatant were then repeated twice, but with the centrifugation time shortened to 3 minutes. 20 μL of Eosin Y was added as a counterstain and the tube was shaken to diffuse the stain evenly in the fluid and the sample was allowed to rest for 5 minutes. The process of adding deionised water, centrifuging and removing supernatant was then performed three times using the same protocol as after addition of the Haematoxylin. At the end of this step, the cells are suitably stained.

### REFERENCE

1. Voet, D.; Voet, J. G. “Biochemistry”; 3rd ed., John Wiley & Sons: USA (2004).
2. Brown, R. B.; Audet, J. “Current techniques for single-cell lysis”; J. R. Soc. Interface, 5(Suppl 2): S131-8 (2008).
3. Lee, S.; Tai, Y. “A micro cell lysis device”; Sensors and Actuators, 73: 74-79 (1999).
4. Tsong, T. Y. “Electroporation of cell membranes”; Biophysics J., 60: 297-306 (1991).
5. Dev, S. B. et al. “Medical applications of electroporation”; IEEE Tran. Plasma Science, 28(1): 206-223 (2000).
6. Muller, K. J.; Sukhorukov, V. L.; Zimmermann, U. “Reversible electropermeabilization of mammalian cells by high-intensity, ultra-short pulses of submicrosecond duration”; J. Membrane Biol., 184(2): 161-170. (2001)
7. Neumann, E. “Electric field-induced structural rearrangements in biomembranes”; Studia Biophysic, 130(1-3): 139-143 (1989).
8. Hu, Q. et al. “Molecular dynamics analysis of high electric pulse effects on bilayer membranes containing DPPC and DPPS”; IEEE Trans. Plasma Science, 34(4): 1405-1411 (2006).
9. Moldovan, D.; Pinisetty, D.; Devireddy, R. V. “Molecular dynamics simulation of pore growth in lipid bilayer membranes in the presence of edge-active agents”; Applied Physics Letters, 91(20): 204104-1-3 (2007).
10. Joshi, R. P.; Shoenbach, K. H. “Electroporation dynamics in biological cells subjected to ultrafast electrical pulses: a numerical simulation study”; Physical Review E, 62(1): 1025-1033 (2000).

1. A membrane disruption device comprising:
a sample container including a sample-containing space defined by a containing surface and configured for containing a cellular-material comprising fluid, wherein the sample-containing space includes at least one membrane disruption space;
an electrode assembly including a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space;
wherein, when the device is disposed in an orientation for containing a cellular material-comprising fluid, each one of the first electrode portion and the second electrode portion, independently, either: (i) defines a lowermost portion of a containing surface portion of the sample container, and the lowermost portion is disposed in a vertical plane orthogonally intersecting the longitudinal axis of the sample-containing space, or (ii) is disposed, relative to a lowermost portion of a containing surface portion of the sample container, vertically above the lowermost portion by a distance of less than one (1) μm, wherein the lowermost portion is disposed in a vertical plane orthogonally intersecting the longitudinal axis of the sample-containing space.

2. The device of claim 1, wherein, each one of the first electrode portion and the second electrode portion is co-operatively disposed relative to the sample container such that, when the sample-containing space is containing its maximum capacity of a cellular material-comprising fluid, each one of the first electrode portion and the second electrode portion is disposed vertically below an upper surface portion of the contained cellular material-comprising fluid by a distance of at least 30 μm.

3. The device of claim 1, wherein each one of the first electrode portion and the second electrode portion is disposed within the membrane disruption space.

4. The device of claim 1, wherein, when the electrode assembly is coupled to a power source such that an electric potential difference is effected between the first electrode portion and the second electrode portion, the electric field is generated between the first electrode portion and the second electrode portion and the membrane disruption of a cell is effected when the cell is disposed between the first electrode portion and the second electrode portion.

5. The device of claim 1, wherein the sample container includes a microchannel, and wherein the sample-containing space is defined within the microchannel.

6. The device of claim 5, wherein the microchannel includes a minimum width, measured horizontally in a plane orthogonal to the axis of the microchannel, of between 40 μm and 10 mm.

7. (canceled)

8. The device of claim 1, wherein the first electrode portion is spaced apart from the second electrode portion by a closest spacing distance of less than 100 μm.

9. (canceled)

10. The device of claim 5, wherein the microcharmel is defined in a cavity provided on a substrate, and wherein each one of the first and second electrode portions is embedded in the substrate.

11. (canceled)

12. (canceled)

13. (canceled)

14. (canceled)

15. (canceled)

16. (canceled)

17. (canceled)

18. (canceled)

19. (canceled)

20. The device of claim 1, further comprising a power supply, wherein, when the electrode assembly is electrically coupled to the power supply such that an electrical potential difference is effected between the first electrode portion and the second electrode portion, a pulsed electric field is generated within the membrane disruption space.

21. (canceled)

22. The device of claim 1, wherein the electrode assembly includes an interdigitated electrode structure including an operative first electrode section and an operative second electrode section, wherein each portion of the operative first electrode section is a first electrode portion and each portion of the operative second electrode section is a second electrode portion.

23. The device of claim 22, wherein the sample container includes a microchannel, wherein the operative first and second electrode sections are disposed on opposite sides of the microchannel.

24. The device of claim 23, wherein the operative first and second electrode sections are substantially co-planar.

25. (canceled)

26. (canceled)

27. The device of claim 22, wherein the width of each of the digits of the operative first and second electrode sections is 10 μm or less.

28. (canceled)

29. (canceled)

30. (canceled)

31. A method of effecting a membrane disruption of a cell using a device of claim 1, comprising:
loading a cell sample including a cell into the sample container;
and applying an electric field to the cell sample.

32. (canceled)

33. (canceled)

34. (canceled)

35. The method of claim 31, wherein a buffer is loaded at a fluidic port and is flowed to the membrane disruption space prior to cell sample loading.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. A membrane disruption device comprising:
a sample container including a sample-containing space defined by a containing surface and configured for containing a cellular-material comprising fluid, wherein the sample-containing space includes at least one membrane disruption space;
an electrode assembly including a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space; and
at least one supply port, wherein each one of the at least one supply port is disposed in fluid communication with the membrane disruption space for effecting supply of material into the membrane disruption space from vertically above the sample-containing compartment.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The device of claim 47, wherein the supply port is fluidly coupled to the membrane disruption space with a fluid passage including a fluid passage axis, and wherein the distance along the fluid passage axis is less than 1.1 millimetres.

64. The device of claim 47, wherein the supply port is configured for any one of effecting supply of cell sample into the membrane disruption space or effecting supply of fluid into the membrane disruption space.

65. A method for staining a cellular sample for visualisation in microfluidic applications comprising:
a) combining a cell sample with a stain; and
b) effecting centrifugal separation of the stained cell-sample and removing at least a fraction of the resulting supernatant.

66. The method of claim 65, further comprising:
(a.1) diluting the stained cell-sample prior to the effecting of centrifugal separation.

67. The method of claim 66, wherein (a.1) and (b) are repeated one or more times.

68. The method of claim 65, further comprising:
combining the stained cell sample with a counterstain;
effecting centrifugal separation of the counterstained cell sample and removing at least a fraction of the resulting supernatant.

69. (canceled)

70. (canceled)

71. (canceled)

72. (canceled)

73. A membrane disruption device comprising:
a sample container including an internal surface defining a sample-containing space configured for containing a cellular-material comprising fluid, wherein the sample-containing space includes at least one membrane disruption space;
an electrode assembly including a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space;
wherein the internal surface includes the first and the second electrode portions.

74. (canceled)

75. A membrane disruption device comprising:
a device substrate including a cavity, wherein a sample-containing space is defined within the cavity, and wherein the sample-containing space is configured for containing a cellular-material comprising fluid, wherein the sample-containing space includes at least one membrane disruption space;
an electrode assembly including a first electrode portion spaced apart from a second electrode portion, wherein, when the electrode assembly is coupled to a power source which effects an electric potential difference between the first electrode portion and the second electrode portion, an electric field is generated within the membrane disruption space and effects membrane disruption of a cell of a cellular material-comprising fluid disposed within the membrane disruption space;
wherein the first and the second electrode portions are embedded in the device substrate.
